(19) United States
(12) Patent Application Publication
(10) Pub. No.: US 2007/0059269 A1
(43) Pub. Date: Mar. 15, 2007

(54) Administration of 8-hexadecene-1,16-dicarboxylic acid for promoting cohesion of the epidermal horny layer

(75) Inventors: Dominique Bernard, Paris (FR); Yann Mahe, Sainte Genevieve Des Bois (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/505,477

(22) Filed: Aug. 17, 2006

Related U.S. Application Data
(60) Provisional application No. 60/716,127, filed on Sep. 13, 2005.

(30) Foreign Application Priority Data
Aug. 17, 2005 (FR) 05/52526

(51) Int. Cl. A61K 8/36

(52) U.S. Cl. 424/70.22

(57) ABSTRACT

A regime or regimen for promoting cohesion and/or organization of the epidermal horny layer, notably for promoting homogeneity and/or clarity of the complexion, for improving the homogeneity and/or the staying power of makeup compositions on the skin or the lips, for preventing impairment of the horny layer induced by a cosmetic or dermatological active agent, or for promoting cohesion of the horny layer in the preparation of reconstructed epidermis and/or reconstructed skin, entails administering to an individual in need of such treatment, for such period of time as required to elicit the desired effect, a cosmetic/dermatological composition which contains up to 8% by weight of 8-hexadecene-1,16-dicarboxylic acid, formulated into a physiologically acceptable medium therefor.

### CROSS-REFERENCE TO PRIORITY/PROVISIONAL APPLICATIONS

This application claims priority under 35 U.S.C. § 119 of FR 05/52526, filed Aug. 17, 2005 and of U.S. Provisional Application No. 60/716,127, filed Sep. 13, 2005, each hereby expressly incorporated by reference and each assigned to the assignee hereof.

### BACKGROUND OF THE INVENTION

1. Technical Field of the Invention

The present invention especially relates to enhancing the appearance and cohesion of the horny layer and its applications, in particular, in the fields of caring for and/or making up the skin and the lips.

This invention also especially relates to the non-therapeutic use of 8-hexadecene-1,16-dicarboxylic acid in or for the preparation of a composition, as an agent for promoting good organization and cohesion of the horny layer and thus especially for promoting the homogeneity and/or clarity of the complexion, for improving the homogeneity and/or staying power of makeup compositions on the skin or the lips, for preventing impairment of the horny layer induced by a cosmetic or dermatological active agent, or for promoting the strength and cohesion of the horny layer in the preparation of reconstructed epidermis and/or reconstructed skin.

Preferably, the 8-hexadecene-1,16-dicarboxylic acid is present in the subject compositions in an amount ranging from 0.00001% to 8% by weight: for these applications, 8-hexadecene-1,16-dicarboxylic acid is effective even for concentrations of less than 0.005% by weight relative to the total weight.

The present invention also relates to makeup compositions comprising at least 8-hexadecene-1,16-dicarboxylic acid and characterized by a particular form, and also to cosmetic or dermatological compositions combining (a) at least 8-hexadecene-1,16-dicarboxylic acid and (b) at least one other acid selected from lactic acid, glycolic acid and ascorbic acid, in a quantitative ratio ranging from 0.005:1 to 0.05:1 and preferably a ratio of 0.01:1.

This invention also relates to cosmetic processes (regime or regimen) useful for improving the surface appearance and/or the comfort of the skin or the lips or the complexion of the skin by administering said compositions.

2. Description of Background and/or Related and/or Prior Art

Human skin consists of two compartments, namely, a deep compartment, the dermis, and a surface compartment, the epidermis.

The dermis provides the epidermis a solid support. It is also the epidermis' nourishing factor. It consists mainly of fibroblasts and of an extracellular matrix that is itself composed mainly of collagen, elastin and a substance known as ground substance, which are synthesized by the fibroblasts. Leukocytes, mastocytes and tissue macrophages are also found therein. The dermis also contains blood vessels and nerve fibers.

Natural human epidermis is composed mainly of three types of cell: keratinocytes, which form the vast majority, melanocytes and Langerhans cells. Each of these cell types contributes, by virtue of its intrinsic functions, towards the essential role played in the body by the skin.

The epidermis is conventionally divided into a basal layer of keratinocytes that constitutes the germinative layer of the epidermis, a layer known as the spiny layer, consisting of several layers of polyhedral cells arranged on the germinative cells, a layer known as the granular layer, consisting of flattened cells containing distinct cytoplasmic inclusions, keratohyalin grains, and finally an upper layer known as the horny layer (or stratum corneum), consisting of corneocytes, which are anuclear mummified cellular structures derived from the keratinocytes.

The corneocytes are mainly composed of a fibrous matrix containing cytokeratins, surrounded by a very strong structure 15 nm thick, known as the horny or hornified envelope. The stacking of these corneocytes constitutes the horny layer, which is the outermost layer and is responsible for the barrier function and the appearance of the epidermis.

It is known that good cohesion and organization of the horny layer are partly associated with the development of the horny envelope, which is controlled especially by enzymes of the transglutaminase family that utilize a set of “precursor” proteins as substrates. The elasticity and solidity of the horny layer are established through a network especially between the horny envelope, the corneodesmosomes and the intermediate cytokeratin filaments.

However, this good organization of the horny layer of the skin or the lips may be impaired or modified by environmental factors such as irritants or pollutants (detergents, pollution, cigarette smoke, etc.), mechanical stresses (rubbing, abrasion or shaving), thermal or climatic imbalance (cold, dryness or radiation), xenobiotic imbalance (microorganisms or allergens), cosmetic or dermatological chemical treatments (scrubbing or anti-acne treatment, etc.) or treatment with physiological factors (age, stress, etc.).

This impairment of the horny layer or rupture of the continuity of the surface of the skin or of the lips may have a repercussion on the homogeneity of their surface, in particular the homogeneity and/or clarity of the complexion of the skin. These variabilities in cohesion and/or structuring of the horny layer may also modify the microrelief or the physicochemical properties of the stratum corneum, leading to poorer staying power of the care compositions and/or of makeup applied to the skin or the lips and consequently to non-uniform distribution of makeup that may give rise to unsightly marks.

Moreover, these variations in the cohesion and/or structuring of the horny layer may participate in the development of pathological disorders such as:
hyperkeratosis, characterized by a thickened horny layer and by abnormal desquamation (e.g., xerosis, ichthyosis, reactional hyperkeratosis, etc.);
leukokeratosis, characterized by transdifferentiation or metaplasia, on the malpighian or non-malpighian but normally non-hornified mucous membranes, which become hornified (e.g., leukokeratosis of the neck of the womb during prolapse, oral leukokeratosis, keratosic benign tumoral lesions of the malpighian mucous membranes, etc.);
cutaneous trophic disorders, characterized, conversely, by thinning of the epidermis and in particular of the horny layer, which is reflected by excessive fragility of the cutaneous covering (e.g., cutaneous trophic disorders of the lower limbs in the case of patients with vascular pathologies such as varicose veins or arteriopathies; cutaneous trophic disorders in the context of an algodystrophic syndrome; trophic disorders following abnormal cicatrization, etc.).

It is also known that the three-dimensional organization and cohesion of the horny layer are important steps in the processes for preparing reconstructed epidermis and/or reconstructed skin, which often indicate their good maturation and quality.

It will thus be appreciated from the foregoing text that there is a need for products capable of promoting and/or restoring good organization and installation of the horny envelopes, which is a guarantee of homogeneity and cohesion of the horny layer at the surface of the skin and the lips.

To promote the cohesion of the horny layer, the final step of its installation may especially be promoted. The term “step of installation of the horny layer” means the process during which the protein precursors produced by the keratinocytes are crosslinked especially via the action of transglutaminase and are insolubilized, these proteins also being able to be insolubilized via bonding with certain ceramides to form hydrophobic structures.

### SUMMARY OF THE INVENTION

It has now been shown, unexpectedly, that 8-hexadecene-1,16-dicarboxylic acid at a low concentration that is not irritant to the skin can satisfy this need. Specifically, it has now been shown that 8-hexadecene-1,16-dicarboxylic acid at low concentration induces the expression of proteins involved in the cohesion of the horny layer (e.g., LEP16) or intercorneocytic cohesion (corneodesmosine).

Similar or complementary results have also been obtained with ascorbic acid, lactic acid and glycolic acid. In this particular case of these compounds, a concentration 10 times higher than that of 8-hexadecene-1,16-dicarboxylic acid is, however, necessary to obtain an equivalent activity.

LEP16 is one of the members of the LEP (Late Envelope Protein) family which appear late in the formation of the horny envelope (Marshall D. et al., PNAS, 2001, Vol. 98, No. 23, 13031-13036); these proteins play a role in the matrix-envelope interaction during correct formation of the horny envelope and of the squama.

The LEPs have recently been described as novel transglutaminase substrates, which are known for their involvement in the formation of the horny envelope by bridging between each other and numerous proteins, the main ones of which are involucrine, loricrine, elafine, cystatins, pancornulins (or SPR: Small Proline Rich), cytokeratins, desmoplakins I and II, desmogleins and corneodesmosine.

The use of 8-hexadecene-1,16-dicarboxylic acid is thus advantageous as a care agent for promoting and/or restoring cohesion of the horny layer and thus for ensuring good homogeneity of the surface of the skin and the lips, which favors a better aesthetic result of makeup compositions (homogeneity and staying power); the use of this acid is also advantageous for the formulation of pharmaceutical compositions for treating skin pathologies associated with abnormally low hornification of the horny layer.

It is also advantageous in processes for preparing reconstructed epidermis and/or reconstructed skin or for good differentiation and maturation of hair follicles in survival.

### DETAILED DESCRIPTION OF BEST MODE AND SPECIFIC/PREFERRED EMBODIMENTS OF THE INVENTION

Hexadecene-1,16-dicarboxylic acid or 9-octadecenedioic acid is a compound that is predominantly found in cis form, obtained by biofermentation of oleic acid in the presence of a mutant yeast of the species Candida. It especially has bleaching and anti-microbial properties making it possible to envisage its use in depigmenting, deodorizing, anti-dandruff and anti-acne products, as described by J. W. Wiechers et al. in Cosmetics & Toiletries, Vol. 117, No. 7, p. 55-68 (July 2002) and in SÖFW Journal, 128, pp. 2-8 (2002).

It has also been proposed in DE-103,05,965 to use it as an antioxidant in cosmetic compositions for rejuvenating or revitalizing the skin, at concentrations of from 0.005% to 20% by weight.

This compound is also described as a chemical scrubbing agent (WO 2005/089 707) at concentrations of at least 10%.

However, to the knowledge of the present inventors, it has never been described or suggested to use 8-hexadecene-1,16-dicarboxylic acid as a care agent for improving and/or restoring the cohesion of the horny layer and thus for improving the homogeneity of the complexion, improving the homogeneity and/or staying power of makeup compositions on the skin or the lips, preventing impairment of the horny layer induced with a cosmetic or dermatological active agent, for instance a keratolytic or anti-acne agent, or promoting cohesion and good organization of the horny layer in the preparation of reconstructed epidermis and/or reconstructed skin.

In particular, 8-hexadecene-1,16-dicarboxylic acid is present in the subject compositions at low concentration, i.e., in an amount necessary to obtain the desired effect, i.e., a care effect intended to improve and/or restore the cohesion and good organization of the horny layer. The concentration of 8-hexadecene-1,16-dicarboxylic acid will be adapted by one skilled in the art to obtain, preferably, a concentration of free acid that is strictly less than 0.005%. Thus, if the 8-hexadecene-1,16-dicarboxylic acid is partially salified or esterified, its total concentration in the composition may be higher; this is likewise the case if the 8-hexadecene-1,16-dicarboxylic acid is provided in a sustained-release form, for example in a vector, for instance nanocapsules or in a patch, which will gradually provide the effective amount to the site of action.

The invention thus relates to the non-therapeutic use of 8-hexadecene-1,16-dicarboxylic acid in or for the preparation of a composition, as an agent for promoting the cohesion and good organization of the horny layer.

For these applications, 8-hexadecene-1,16-dicarboxylic acid may be present in a concentration ranging from 0.00001% to 8% or even at less than 5×10⁻³% by weight relative to the total weight of the composition.

More preferentially, 8-hexadecene-1,16-dicarboxylic acid is present in the subject compositions in an amount ranging from 0.0001% to 0.0005% by weight relative to the total weight.

The 8-hexadecene-1,16-dicarboxylic acid according to the invention may be in cis form, in trans form or as a mixture of these two forms. It is especially commercially available from the company Uniqema under the trademark Arlatone Dioic DCA.

The 8-hexadecene-1,16-dicarboxylic acid present in the subject compositions will especially be intended:
to promote the homogeneity and/or clarity of the complexion;
to promote the homogeneity and/or staying power of a makeup composition applied to the skin or the lips;
to promote the re-epithelialization and/or regeneration of the skin or the lips; in particular in the case of skin and/or lips impaired by external attack (cold, mechanical rubbing, etc.), burns, exposure to UV or scrubbing treatments;
to promote the re-epithelialization and/or regeneration of the contour of the hair root, by ensuring better cohesion at the infundibular junction between the epidermis and the hair stem. The compositions or the 8-hexadecene-1,16-dicarboxylic acid may thus promote the regrowth and the quality of the hair and/or reduce hair loss, optionally in combination with other active agents. They may also be used to promote the growth of the eyelashes;
to promote the re-epithelialization and/or regeneration of the nails in particular at the cuticle. By promoting the hornification of the nail, treatment or prevention of the phenomenon of brittle nails is targeted in particular;
to improve the comfort and strength of skin, scalp or lips that has(have) been impaired by at least one condition selected from cold, UV radiation and mechanical rubbing (e.g., shaving, epilation, etc.);
to prepare the skin for the action of scrubbing or exposure to sunlight.

8-Hexadecene-1,16-dicarboxylic acid is also advantageously administered according to the invention to unify the color of a suntan.

For all these applications, 8-hexadecene-1,16-dicarboxylic acid is active at a low concentration, beginning at 0.00001%, but it may be employed at higher concentrations; this amount may vary, for example, from 0.00001% to 8% or to 5% by weight relative to the total weight of the composition, preferably greater than or equal to 0.0001%; amounts suited to the application of the invention will especially be from 0.0001% to 0.005% or 0.0001% to 0.0005% by weight relative to the total weight of the composition.

Advantageously, this amount will not exceed 8% by weight relative to the total weight of the composition and preferably will not exceed 5% by weight relative to the total weight of the composition.

The invention also relates to the non-therapeutic use of 8-hexadecene-1,16-dicarboxylic acid in or for the preparation of a composition, as an agent for preventing impairment of the horny layer induced by the action of a cosmetic or dermatological active agent present in the said composition, and/or to promote its restoration.

The compositions according to the invention may effectively be a cosmetic or dermatological composition and may contain at least one active agent with a side effect that is potentially deleterious to the strength and the cohesion of the horny layer. This may be, for example, a cosmetic or dermatological active agent selected from the group of keratolytic or desquamating agents, anti-seborrhoeic and/or anti-acne agents, and comedolytic agents, and mixtures thereof.

Examples of keratolytic agents that may especially be mentioned include: α-hydroxy acids, for instance glycolic acid, lactic acid, dioic acid, malic acid, citric acid, tartaric acid and mandelic acid, or derivatives thereof; β-hydroxy acids, for instance salicylic acid and derivatives thereof; α-keto acids, for instance ascorbic acid or vitamin C and derivatives thereof; P-keto acids; retinoids, for instance retinol and esters thereof, retinal, retinoic acid and derivatives thereof, and those described in FR-A-2-570,377, EP-A-199,636, EP-A-325,540 and EP-A-402,072.

The term “desquamating agent” means any compound capable of acting:

either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea and certain derivatives thereof; gentisic acid; oligofucoses; cinnamic acid; extract of Saphorajaponica; resveratrol; detergents and certain jasmonic acid derivatives;

and/or on the activity of the enzymes involved in degradation of corneodesmosomes, such as stratum corneum chymotryptic enzyme (SCCE), or even other proteases (trypsin-like, chymotrypsin-like, cathepsin D) and also other categories of hydrolases (e.g., glycosidases, ceramidases). Exemplary are agents for chelating mineral salts: EDTA; N-acyl-N,N′,N′-ethylenediaminetriacetic acid; aminosulfonic compounds and in particular (N-2-hydroxyethylpiperazine-N-2-ethane)sulfonic acid (HEPES); 2-oxothiazolidine-4-carboxylic acid (procysteine) derivatives; a-amino acid derivatives of the glycine type (such as described in EP-0 852 949 and sodium methylglycinediacetate marketed by BASF under the trademark Trilon M); honey; sugar derivatives such as O-octanoyl-6-D-maltose and N-acetylglucosamine; urea or certain derivatives thereof.

Examples of anti-seborrhoeic and/or anti-acne agents that are especially representative include:
retinoids, and in particular retinol;
sulfur and sulfur derivatives;
zinc salts such as zinc lactate, gluconate, pidolate, carboxylate, salicylate and/or cysteate;
selenium chloride;
vitamin B6 or pyridoxine;
mixture of capryloyl glycine, sarcosine and extract of Cinnamomum zeylanicum marketed especially by SEPPIC under the trademark Sepicontrol A5®;
an extract of Laminaria saccharina marketed especially by SECMA under the trademark Phlorogine®;
an extract of Spiraea ulmaria marketed especially by Silab under the trademark Sebonormine®;
plant extracts from the species Amica montana, Cinchona succirubra, Eugenia caryophyllata, Humulus lupulus, Hypericum perforatum, Mentha piperita, Rosmarinus officinalis, Salvia oficinalis and Thymus vulgaris, all marketed, for example, by Maruzen;
an extract of Serenoa repens marketed especially by Euromed;
plant extracts of the genus Silybum; and
extracts of Eugenia caryophyllata containing eugenol and eugenyl glucoside;
sulfonic acids such as those described in EP-0-728,474 (L'Oréal).

Examples of comedolytic agents that are especially representative include: salicylic acid and HEPES.

The presence of 8-hexadecene-1,16-dicarboxylic acid at low concentration in the cosmetic or dermatological composition according to the invention will make it possible to prevent and/or reduce the side effect that is potentially deleterious on the cohesion and organization of the horny layer by a cosmetic or dermatological active agent present in the said composition.

The present invention also features the use of 8-hexadecene-1,16-dicarboxylic acid for the preparation of a dermatological composition for treating skin complaint, condition or afflictions associated with abnormal formation of the horny layer, in particular hyperkeratosis, leukokeratosis, parakeratosis and cutaneous trophic disorders.

8-Hexadecene-1,16-dicarboxylic acid is present in the composition in an amount required to obtain the desired effect, i.e., an improvement and/or restoration of maturation and/or cohesion of the horny layer.

As indicated previously, the concentration of 8-hexadecene-1,16-dicarboxylic acid will be adapted by one skilled in the art as a function of the form in which it is present in the composition, to obtain an effective amount. This amount may range from 0.00001% to 5% or from 0.00001% to 8% by weight relative to the total weight of the composition; amounts suited to the use of the invention will especially be from 0.0001% to 0.005% or from 0.0001% to 0.0005% by weight relative to the total weight of the composition.

Advantageously, this amount will not exceed 8% by weight relative to the total weight of the composition and preferably will not exceed 5% by weight relative to the total weight of the composition.

Advantageously, the 8-hexadecene-1,16-dicarboxylic acid is combined in the composition of the invention with at least one other acid selected from lactic acid, glycolic acid and ascorbic acid, and mixtures thereof, for which it has been shown that additional or complementary effects on maturation of the horny layer, intercorneocytic cohesion and/or re-epithelialization are realized.

The present invention thus also features the administration of at least one compound selected from lactic acid and glycolic acid as an agent for promoting maturation and/or cohesion of the horny layer.

When 8-hexadecene-1,16-dicarboxylic acid and the other agents are used in combination, this also makes it possible to reduce the effective amounts of each active agent present in the composition.

The compositions according to the invention may also contain urea, which will promote the final horny-layer-improving activity. 8-Hexadecene-1,16-dicarboxylic acid and at least one other acid and/or urea, in amounts of from 0.01% to 10% by weight, may be used simultaneously, separately, staggered and/or sequentially over time.

In particular, 8-hexadecene-1,16-dicarboxylic acid and the other acid selected from lactic acid, glycolic acid and ascorbic acid, each of these acids possibly being present in the composition in amounts of from 0.01% to 10% by weight, and mixtures thereof are present in the composition in a quantitative ratio ranging from 0.005:1 to 0.05:1 and preferably in a ratio of 0.01:1.

The present invention also features a cosmetic regime or regimen for unifying the color of a suntan, comprising the application to the skin of 8-hexadecene-1,16-dicarboxylic acid before or after exposure to the sun.

The amount of 8-hexadecene-1,16-dicarboxylic acid may range from 0.00001% to 5% or from 0.00001% to 8% by weight relative to the total weight of the composition, and amounts suited to the use of the invention will especially be from 0.0001% to 0.005% or from 0.0001% to 0.0005% by weight relative to the total weight of the composition.

The compositions according to the invention may be for cosmetic or dermatological applications. When it is a dermatological composition, it will especially be a scrubbing composition or an anti-acne composition.

Preferentially, the compositions of the invention are for cosmetic use for improving the surface homogeneity of the skin or the lips, in particular the homogeneity of the complexion. It is in particular a composition for caring for and/or making up the skin or the lips, which may especially be in the form of a skincare base, a care cream (day cream, night cream or anti-wrinkle cream), a makeup base, or a tinted care cream, or in the form of a foundation of fluid, semi-solid or solid consistency.

It may be in any galenical form normally used in cosmetics and dermatology, suited to the oral or topical route, preferentially the topical route.

For oral administration, in particular “oral cosmetic” administration, it may especially be in the form of wafer capsules, gel capsules, sugar-coated tablets, granules, chewing gum, gels, drinkable syrups, tablets or any other form known to those skilled in the art.

For topical application to the skin, the scalp or the lips, the composition may be in the form of an optionally gelled aqueous, aqueous-alcoholic or oily solution, an emulsion of liquid or semi-liquid consistency of the milk type, obtained by dispersing a fatty phase in an aqueous phase (O/W) or conversely (W/O), a triple emulsion (W/O/W or O/W/O) or a suspension or emulsion of soft, semi-solid or solid consistency of cream or gel type, a liquid, pasty or solid anhydrous product, or, alternatively, microemulsions, microcapsules, microparticles or a vesicular dispersion of ionic type (liposomes or oleosomes) and/or nonionic type (niosomes) and/or a dispersion of nanospheres.

A composition in mousse form or in spray or aerosol form then comprising a pressurized propellant may also be administered.

The composition may thus be in the form of a lotion, serum, milk, O/W or W/O cream, gel, ointment, pomade, powder, balm, patch, impregnated pad, soap, bar or mousse. It may also be in the form of a lipstick, a lip paste or a lip gloss, a powder, a solid or semi-solid foundation, or a mascara to be applied to the hair and/or the eyelashes. It may especially be in the form of a patch or transdermal delivery device, intended for action at the site of application or remote action by diffusion of the active agents into the cutaneous compartment.

When the composition is an emulsion, the proportion of the fatty phase of the composition under consideration may range, for example, from 5% to 80% by weight and especially from 5% to 50% by weight relative to the total weight of the composition.

The aqueous phase may consist essentially of water or may comprise a mixture of water and of water-miscible organic solvent (miscibility in water of greater than 50% by weight at 25° C.), for instance lower monoalcohols containing from 1 to 5 carbon atoms such as ethanol or isopropanol, glycols containing from 2 to 8 carbon atoms such as propylene glycol, ethylene glycol, 1,3-butylene glycol or dipropylene glycol, C₃-C₄ ketones and C₂-C₄ aldehydes.

This aqueous phase (water and optionally the water-miscible organic solvent) may be present in the base composition in a content ranging from 1% to 95% by weight, especially ranging from 3% to 80% by weight and in particular ranging from 5% to 60% by weight relative to the total weight of the base composition.

The fatty phase of the composition may contain fatty or oily compounds, and optionally silicone or non-silicone waxes, gums and pasty fatty substances of plant, animal, mineral or synthetic origin.

Examples of oils that are representative include:
hydrocarbon-based oils of animal origin, such as perhydrosqualene;
hydrocarbon-based oils of plant origin, such as liquid triglycerides of fatty acids containing from 4 to 10 carbon atoms and the liquid fraction of shea butter;
synthetic esters and synthetic ethers, especially of fatty acids, for instance oils of formulae R¹COOR² and R′OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils, for instance those described in JP-A-2 295 912;
silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyidiphenyltrisiloxanes, 2-phenylethyltrimethyl siloxysilicates and polymethylphenylsiloxanes; and
mixtures thereof.

The waxes may be hydrocarbon-based waxes, silicone waxes and/or fluoro waxes, optionally comprising ester or hydroxyl functions. They are especially of natural origin.

The wax may represent from 0.01% to 10% by weight and especially from 0. 1% to 5% by weight relative to the total weight of the composition.

As waxes that may be included in the compositions of the invention, exemplary are beeswax, carnauba wax, candelilla wax, paraffin wax, microcrystalline waxes, ceresin or ozokerite; synthetic waxes, for instance polyethylene wax or Fischer-Tropsch wax, and silicone waxes, for instance alkyl or alkoxy dimethicones containing from 16 to 45 carbon atoms.

Among the pasty compounds that may be included in the compositions according to the invention, exemplary are lanolins and lanolin derivatives, for instance acetylated lanolins, oxypropylenated lanolins or isopropyl lanolate, and mixtures thereof. Esters of fatty acids or of fatty alcohols may also be included, especially those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be used include hydrogenated castor oil derivatives, such as Thixinr® from Rheox.

Also exemplary are polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) marketed by the Japanese company Kokyu Alcohol Kogyo.

Also exemplary are pasty silicone compounds such as high molecular weight polydimethylsiloxanes (PDMSs) and in particular those containing pendent chains of the alkyl or alkoxy type containing from 8 to 24 carbon atoms, and having a melting point of 20-55° C., for instance stearyl dimethicones, especially those marketed by Dow Corning under the trademarks DC2503® and DC25514®, and mixtures thereof.

The compositions may also contain adjuvants that are common in cosmetics, such as fillers, dyestuffs, hydrophilic or lipophilic cosmetic active agents, thickeners, emulsifiers, hydrophilic or lipophilic gelling agents, surfactants, moisturizers, softeners, sequestrants, fragrances, neutralizers, preservatives, antioxidants, UV-screening agents, bactericides, trace elements, odor absorbers and pH regulators, and mixtures thereof.

The amounts of these various adjuvants are those conventionally used in the field under consideration, for example from 0.01% to 20% of the total weight of the composition. Depending on their nature, these adjuvants may be introduced into the fatty phase or into the aqueous phase.

In any case, these adjuvants, and the proportions thereof, will be selected such as not to adversely affect the desired properties according to the invention.

As emulsifiers and co-emulsifiers that may be used, representative are, for example, O/W emulsifiers such as fatty acid esters of polyethylene glycol, especially PEG-100 stearate, and fatty acid esters of glycerol, such as glyceryl stearate, and also W/O emulsifiers available, for example, under the trademarks, Abil WE09, Abil EM90 and Abil EM97 from the company Degussa Goldschmidt, or the mixture of acetyl ethylene glycol stearate and of glyceryl tristearate marketed by Guardian under the trademark Unitwix.

As hydrophilic gelling agents that may be included, exemplary are, in particular, carboxyvinyl polymers (carbomer), acrylic copolymers such as acrylate/alkylacrylate copolymers, polyacrylamides, polysaccharides, natural gums and clays, and lipophilic gelling agents that are exemplary include modified clays, for instance bentones, metal salts of fatty acids, hydrophobic silica and polyethylenes.

The term “fillers” should be understood as meaning colorless or white, mineral or synthetic particles of any form, which are insoluble in the medium of the composition irrespective of the temperature at which the composition is manufactured.

The fillers may be selected especially from among mineral and organic fillers of any form, platelet-shaped, spherical or oblong, irrespective of the crystallographic form (for example lamellar, cubic, hexagonal, orthorhombic, etc.). Mention may be made of talc, mica, silica, kaolin, starch crosslinked with octenylsuccinic anhydride marketed by National Starch under the name Dry Flo Plus (28-1160); polyamide particles and especially those marketed under the name Orgasol by Atochem; microspheres based on acrylic copolymers, such as those made of ethylene glycol dimethacrylate/lauryl methacrylate copolymer marketed by Dow Corning under the name Polytrap; expanded powders such as hollow microspheres and especially the microspheres marketed under the name Expancel by Kemanord Plast or under the name Micropearl F 80 ED by Matsumoto; polymethacrylate-type powders (PMMA), silicone elastomers and silica powders of the Sunsphere type, silicone resin microbeads such as those marketed under the name Tospearl by Toshiba Silicone; and mixtures thereof.

These fillers may be present in amounts ranging from 0.01% to 20% by weight and preferably from 1% to 10% by weight, relative to the total weight of the base composition.

Dyestuffs that are especially representative include monochromatic pigments, nacres, reflective pigments that do or do not emit a color, interference pigments, liposoluble dyes and water-soluble dyes, and mixtures thereof.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow.

The pigments may be white or colored, mineral and/or organic, and coated or uncoated. Among the mineral pigments that may be mentioned are titanium dioxide, optionally surface-treated, zirconium oxide or cerium oxide, and also iron oxide or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum.

The nacreous pigments may be selected from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the abovementioned type, and nacreous pigments based on bismuth oxychloride.

The pigments may have undergone a surface treatment.

As hydrophilic or lipophilic cosmetic active agents that are suitable for use in the compositions of the invention, cosmetic active agents intended to improve the surface appearance and/or the comfort of the skin, the scalp or the lips, or the complexion of the skin, will especially be used.

This active agent may be selected especially from an agent for stimulating the synthesis of dermal or epidermal macromolecules and/or for preventing their degradation, an agent for stimulating fibroblast or keratinocyte proliferation and/or keratinocyte differentiation, a moisturizer, a depigmenting agent, an agent for promoting coloring of the skin, an anti-pollution agent or free-radical scavenger, a dermo-relaxing agent and a tensioning agent, and mixtures thereof.

Agents for Stimulating the Synthesis of Dermal or Epidermal Macromolecules and/or for Preventing their Degradation:

The cells of the dermis, in particular fibroblasts, produce collagen, elastin and glycoprotein molecules. With the effect of age or under the effect of UV radiation there is a significant reduction in these molecules, and degradation of the collagen and elastin fibers occurs under the effect of collagenase or elastase.

Among the active agents for stimulating the dermal macromolecules or for preventing their degradation, exemplary are those that act:

either on collagen synthesis, such as extracts of Centella asiatica; asiaticosides and derivatives thereof; ascorbic acid or vitamin C and derivatives thereof, such as the salts or esters thereof, in particular 5,6-di-O-dimethylsilyl ascorbate (marketed by Exsymol under the reference PRO-AA), the potassium salt of di-α-tocopheryl-di-ascorbyl phosphate (marketed by Senju Pharmaceutical under the reference Sepivital EPC), magnesium ascorbyl phosphate, sodium ascorbyl phosphate (marketed by Roche under the reference Stay-C 50) and ascorbyl glucoside (marketed by Hayashibara); synthetic peptides such as iamin, the biopeptide CL or palmitoyl oligopeptide marketed by Sederma; peptides extracted from plants, such as the soybean hydrolysate marketed by Coletica under the trademark Phytokine®; plant hormones such as auxins and lignans; the palmitoyl of the pentapeptide lysine-threonine-threonine-lysine-serine marketed especially under the name “Matrixyl” by Sederma; dimethylaminoethanol; extracts of Bupleurum chinensis rhizome, such as the products marketed under the names “Pleurimincyl” and “Lipocare” by Sederma; wheat protein hydrolysates acylated especially with a palmitoyl group, such as the products marketed under the name “Lipacid PVB” by SEPPIC; creatine; coenzyme Q10;

or on elastin synthesis, such as the extract of Saccharomyces cerevisiae marketed by LSN under the trademark Cytovitin®; and the extract of the alga Macrocystis pyrifera marketed by SECMA under the trademark Kelpadelie®; melibiose; soybean proteins;

or on glycosaminoglycan synthesis, such as the product of fermentation of milk with Lactobacillus vulgaris, marketed by Brooks under the trademark Biomin yogourth®; the extract of the brown alga Padina pavonica marketed by Alban Müller under the trademark HSP3®; and the extract of Saccharomyces cerevisiae available especially from the company Silab under the trademark Firmalift® or from the company LSN under the trademark Cytovitin®;

or on fibronectin synthesis, such as the extract of the zooplankton Salina marketed by Seporga under the trademark GP4G®; the yeast extract available especially from the company Alban Müller under the trademark Drieline®; and the palmitoyl pentapeptide marketed by Sederma under the trademark Matrixil®;

or on the inhibition of metalloproteases (matrix metalloproteases or MMPs), such as, more particularly, MMP 1, 2, 3 or 9. Mention may be made of: retinoids and derivatives, oligopeptides and lipopeptides, lipoamino acids, the malt extract marketed by Coletica under the trademark Collalift®; extracts of blueberry or of rosemary; lycopene; isoflavones, their derivatives or plant extracts containing them, in particular extracts of soybean (marketed, for example, by lchimaru Pharcos under the trademark Flavosterone SB®), of red clover, of flax, of kakkon, or of sage;

or on the inhibition of serine proteases such as leukocyte elastase or cathepsin G. Mention may be made of: the peptide extract of legume seeds (Pisum sativum) marketed by LSN under the trademark Parelastyl®; and heparinoids and pseudodipeptides such as {2-[acetyl(3-trifluoromethylphenyl)amino]-3-methylbutyrylamino}acetic acid.

Among the active agents that stimulate fillagrin and keratins, especially representative are the extract of lupin marketed by Silab under the trademark Structurine®; the extract of beech Fagus sylvatica buds marketed by Gattefosse under the trademark Gatuline®; and the extract of the zooplankton Salina marketed by Seporga under the trademark GP4G®.

Preferably, the agents for stimulating the synthesis of dermal or epidermal macromolecules and/or for preventing their degradation are selected from extracts of Centella asiatica, ascorbic acid and derivatives thereof, peptides extracted from plants, such as the soybean hydrolysate marketed by Coletica under the trademark Phytokine®, the extract of Saccharomyces cerevisiae marketed by LSN under the trademark Cytovitin®; the extract of the brown alga Padina pavonica marketed by Alban Muller under the trademark HSP3®; retinoids and derivatives; extracts of rosemary; the peptide extract of legume seeds (Pisum sativum) marketed by LSN under the trademark Parelastyl®; {2-[acetyl(3-trifluoromethylphenyl)amino]-3-methylbutyrylamino}acetic acid; extract of lupin; and mixtures thereof.

Preferred examples of compounds for reinforcing and/or repairing the barrier function that may be used include ceramides, precursors or derivatives, ceramide synthesis stimulators, ceramidase inhibitors, sphingomyelinases, sphingomyelinase stimulators, AGEs, gamma-linolenic acid, omega-3 or omega-6 unsaturated fatty acids, unsaponifiable materials (shea butter, avocado oil, corn oil, etc.), galactolipids, phospholipids, squalane and squalene.

Agents for Stimulating Fibroblast or Keratinocyte Proliferation and/or Keratinocyte Differentiation:

The agents for stimulating fibroblast proliferation that may be included in the compositions according to the invention may be selected, for example, from plant proteins or polypeptides, extracted especially from soybean (for example an extract of soybean marketed by LSN under the name Eleseryl SH-VEG 8® or marketed by Silab under the trademark Raffermine®); and plant hormones such as giberrellins and cytokinins.

The agents for stimulating keratinocyte proliferation that may be included in the compositions according to the invention especially comprise retinoids such as retinol and its esters, including retinyl palmitate; adenosine; phloroglucinol; extracts of walnut cakes marketed by Gattefosse; and extracts of Solanum tuberosum marketed by Sederma.

The agents for stimulating keratinocyte differentiation comprise, for example, minerals such as calcium; a peptide extract of lupin such as the product marketed by Silab under the trademark Structurine®; sodium β-sitosteryl sulfate such as the product marketed by Seporga under the trademark Phytocohesine®; and a water-soluble extract of corn such as the product marketed by Solabia under the trademark Phytovityl®; a peptide extract of Voandzeia substerranea such as the product marketed by Laboratoires Sérobiologiques under the trademark Filladyn LS 9397®; and lignans such as secoisolariciresinol.

As preferred examples of compounds that promote keratinocyte proliferation, mention may be made of capryloylsalicylic acid, Stimoderm®, helichrysum, samphire, lycopene, tomato extracts, Lanablue®, Vitoptine®, extract of mamaku, Structurine®, Nutelline® and caobromine.

Moisturizers:

The term “moisturizer” means:
either a compound acting on the barrier function, in order to keep the stratum corneum moisturized, or an occlusive compound. Exemplary are ceramides, sphingoid-based compounds, lecithins, glycosphingolipids, phospholipids, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;
or a compound that directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides such as the product marketed under the reference Pentavitin, honey, alginates (in particular the product Sobaig PH 154 marketed by Grindsted), cyclic carbonates, N-lauroylpyrrolidonecarboxylic acid or salts thereof, in particular the sodium salt marketed under the reference Nalidone, and N-α-benzoyl-L-arginine;
or a compound that activates the sebaceous glands, such as steroid derivatives (including DHEA, the 7-oxido and/or 17-alkyl derivatives thereof, and sapogenins), methyl dihydrojasmonate and vitamin D and its derivatives.

These compounds may represent from 0.001% to 30% to preferably from 0.01% to 20% of the total weight of the composition according to the invention.

Depigmenting Agents:

The depigmenting or anti-pigmenting agents that may be incorporated into the compositions according to the present invention comprise, for example, the following compounds: kojic acid; ellagic acid; arbutin and its derivatives such as those described in EP-895,779 and EP-524,109; hydroquinone; aminophenol derivatives such as those described in WO 99/10318 and WO 99/32077, and in particular N-cholesteryloxycarbonyl-para-aminophenol and N-ethyloxycarbonyl-para-aminophenol; iminophenol derivatives, in particular those described in WO 99/22707; L-2-oxothiazolidine-4-carboxylic acid or procysteine, and also its salts and esters; calcium D-pantheteine sulfonate; ascorbic acid and its derivatives, especially ascorbyl glucoside; and plant extracts, in particular extracts of liquorice, of mulberry, of skullcap and of Bacopa monieri, without this list being limiting.

Skin-coloring Agents:

These agents may especially be selected from an agent for promoting melanogenesis and an artificial skin-coloring agent, and mixtures thereof.

According to the invention, the term “agent for promoting melanogenesis” especially means:
a melanin biosynthesis substrate;
a melanin biosynthesis stimulator, or biological activators of melanogenesis capable of acting:
   by stimulating melanin synthesis; and/or
   by stimulating the activity or expression of tyrosinase, optionally by increasing the level of intracellular cAMP or by activating protein kinase C, and/or
   by stimulating the transfer of the melanosomes from the melanocytes to the keratinocytes, for example by stimulating the PAR-2 receptors.

These agents for promoting melanogenesis may be present in the composition in an amount of from 0.1% to 15% by weight and preferably in an amount of from 0.5% to 5% of the total weight of the composition.

Examples of “melanin biosynthesis substrates” that are representative include:
L-dopa provided, for example, by extracts of plants such as Vicia faba or Musa sativa;
L-tyrosine and derivatives thereof, or L-dihydrophenylalanine.

As “compounds for stimulating melanin biosynthesis” according to the invention, mention may be made more particularly of the aliphatic or cyclic diols as described in J. Invest. Dermatol., 1998, 110, 4, 428 (for example 5-norbornene-2,2-dimethanol or 3,3-dimethyl-1,2-butanediol); peptides with isoelectric points of between 6 and 11 as described in WO 99/37279 (for example the peptide Asp-Gln-Pro-Leu-Leu-Thr-P in which P is a hydrophobic amino acid such as tryptophan Trp); nucleopeptide complexes as described in WO 98/12212 (for example the complex formed by: puric acid-alanine-histidine-dibromophenylalanine-NH₂); adenosine-1 receptor antagonists (for example 1,3-dimethyl-8-cyclopentylxanthine or 1,3-diisopropyl-8-cyclopentylxanthine) or adenosine-2 receptor agonists (for example 2-[(cyclohexylethyl)amino]adenosine) as described in WO 98/15276; complexes of metal ions such as copper and of peptones such as a protein hydrolysate derived from soybean, collagen or casein, as described in U.S. Pat. No. 5,698,184. These documents are all incorporated herein by reference.

Among the compounds for stimulating tyrosinase activity or expression according to the invention, exemplary are the prostaglandins as described in WO 98/00100; NO activators/cGMP/protein kinase C, for example isosorbide dinitrate/cGMP/protein kinase C (WO 98/11882); or, alternatively, extracts of plants selected from Caesapinia sappan (EP 820 761), Parameria laevigata, Piper cubeba, Sonchus arvensis, Pluchea indica L., Massoia aromatica, Alstonia scholaris, Alycia reindwartii Bl., Cinnamomum sintoc, Arctostaphylos, Chenopodium, Poterium and Gautheria (EP-914,816). These documents are all incorporated herein by reference.

Among the compounds for stimulating tyrosinase expression by increasing the level of intracellular cAMP, especially exemplary are pro-opiomelanocortic peptides; α-MSHs or α-MSH analogues (for example Ac-[D]Phe-α-MSH₁₋₁₃-NH₂); or MC1 R receptor agonists (U.S. Pat. No. 5,683,981 or WO 98/25584), cAMP analogues as described in Biochem. Biophys. Res. Comm., 1987, 145, 719, forskolin (J. Int. Med. Res., 1990, 18, 8C-17C) and xanthine bases (for example caffeine or theophylline).

As compounds for stimulating tyrosinase expression by activation of protein kinase C, exemplary are the diacylglycerols as described in J. Invest. Dermatol., 1995, 105, 5, 687, or psoralenes such as those described in Photodermatol. Photoimmunol. Photomed., 1997, 13, 9. These documents are all incorporated herein by reference.

Finally, compounds for stimulating the transfer of melanosomes from the melanocytes to the keratinocytes by stimulation of the PAR-2 receptors or of the PAR-2 agonists (for example the peptide of composition Ser-Leu-lle-Gly-Art-Leu) have been described in WO 99/04752, which is incorporated herein by reference.

Mention may also be made of the catechin polyphenols described in L'Oréal WO 04/080 380. In particular, catechin, epicatechin, gallocatechin and epigallocatechin, the salts and esters thereof, in monomeric or oligomeric form, and also plant extracts containing them (e.g., extracts of green tea).

Examples of “artificial skin-coloring agents” that may be mentioned include:
a self-tanning agent, i.e., an agent which, when applied to the skin, especially to the face, can produce a tan effect that is more or less similar in appearance to that which may result from prolonged exposure to the sun (natural tan) or under a UV lamp;
an additional coloring agent, i.e., any compound that has particular affinity for the skin, which allows it to give the skin a lasting, non-covering coloration (i.e., that does not have a tendency to opacify the skin) and that is not removed either with water or using a solvent, and that withstands both rubbing and washing with a solution containing surfactants. Such a lasting coloration is thus distinguished from the superficial and transient coloration provided, for example, by a makeup pigment;
   and mixtures thereof.

Examples of self-tanning agents that are especially representative include:
dihydroxyacetone (DHA),
erythrulose, and
the combination of a catalytic system formed from:
manganese and/or zinc oxide salts, and
alkali metal and/or alkaline-earth metal hydrogen carbonates.

The self-tanning agents are generally selected from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in FR-2,466,492 and WO 97/35842, dihydroxyacetone (DHA) and 4,4-dihydroxypyrazolin-5-one derivatives as described in EP-903,342. DHA will preferably be used.

The DHA may be used in free and/or encapsulated form, for example in lipid vesicles such as liposomes, especially described in WO 97/25970.

In general, the self-tanning agent is present in an amount ranging from 0.01% to 20% by weight and preferably in an amount of from 0.1% to 10% of the total weight of the composition.

Examples of additional coloring agents that may be mentioned include plant extracts, for instance sorghum extracts obtained from the whole plant or from the stems, seeds or leaves of the genus Sorghum. The preferred Sorghum species are selected from Sorghum bicolor, Sorghum caudatum, Sorghum nervosum, Sorghum durra, Sorghum vulgare and Sorghum species in combination with Colletotrichum graminicola, for instance those described in FR 02/00251.

Other dyes that allow modification of the color produced by the self-tanning agent may also be used.

These dyes may be selected from synthetic or natural direct dyes.

These dyes may be selected, for example, from red or orange dyes of the fluorane type such as those described in FR-2-840,806. Mention may be made, for example, of the following dyes:
tetrabromofluoresceine or eosin known under the CTFA name: CI 45380 or Red 21;
phloxin B known under the CTFA name: CI 45410 or Red 27;
diiodofluoresceine known under the CTFA name: CI 45425 or Orange 10;
dibromofluoresceine known under the CTFA name: CI 45370 or Orange 5;
the sodium salt of tetrabromofluoresceine known under the CTFA name: CI 45380 (Na salt) or Red 22;
the sodium salt of phloxin B known under the CTFA name: CI 45410 (Na salt) or Red 28;
the sodium salt of diiodofluoresceine known under the CTFA name: CI 45425 (Na salt) or Orange 11;
erythrosine known under the CTFA name: CI 45430 or Acid Red 51;
phloxin known under the CTFA name: CI 45405 or Acid Red 98.

These dyes may also be selected from anthraquinones, caramel, carmine, carbon black, azulene blues, methoxalene, trioxalene, guajazulene, chamuzulene, Bengal rose, cosin 10B, cyanosin and daphinin.

These dyes may also be selected from indole derivatives, for instance the monohydroxyindoles as described in FR-2-651,126 (i.e.,: 4-, 5-, 6- or 7-hydroxyindole) or the dihydroxyindoles as described in EP-B-0-425,324 (i.e.,: 5,6-dihydroxyindole, 2-methyl-5,6-dihydroxyindole, 3-methyl-5,6-dihydroxyindole or 2,3-dimethyl-5,6-dihydroxyindole).

Anti-pollution Agents or Free-radical Scavengers:

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be included in the compositions according to the invention, exemplary are, in particular, vitamin C and its derivatives including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolysed RNA, marketed by Laboratoires Serobiologiques under the trademark CPP LS 2633-12F®, the water-soluble fraction of corn marketed by Solabia under the trademark Phytovityl®, the mixture of extract of fumitory and of extract of lemon marketed under the name Unicotrozon C-49® by Induchem, and the mixture of extracts of ginseng, of apple, of peach, of wheat and of barley, marketed by Provital under the trademark Pronalen Bioprotect®.

As agents for trapping monocyclic or polycyclic aromatic compounds, which may be included in the compositions according to the invention, exemplary are, in particular, tannins such as ellagic acid; indole derivatives, in particular 3-indolecarbinol; extracts of tea, in particular of green tea, extracts of water hyacinth or Eichornia crassipes; and the water-soluble fraction of corn marketed by Solabia under the trademark Phytovityl®.

Finally, as heavy-metal-trapping agents that may be included in the compositions according to the invention, exemplary are, in particular, of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichomia crassipes); and the water-soluble fraction of corn marketed by Solabia under the trademark Phytovityl®.

The free-radical scavengers that may be included in the compositions according to the invention comprise, other than certain anti-pollution agents mentioned above, vitamin E and its derivatives such as tocopheryl acetate; bioflavonoids; coenzyme Q10 or ubiquinone; certain enzymes, for instance catalase, superoxide dismutase and extracts of wheatgerm containing it, lactoperoxidase, glutathione peroxidase and quinone reductases; glutathione; benzylidenecamphor; benzylcyclanones; substituted naphthalenones; pidolates; phytantriol; gamma-oryzanol; guanosine; lignans; and melatonin.

Among these free-radical scavengers, mention is made of the superoxide dismutase mimetics extracted from marrow or melon or else synthesized, and also compounds such as the thermal plankton extract of Vitreoscilla filiformis or its purified LPS, which are capable of inducing endogenous cutaneous MnSODs.

Dermo-relaxing Agents:

The dermo-decontracting or dermo-relaxing agents that may be included in the compositions according to the invention comprise alverine and its salts, manganese gluconate, Diazepam, the hexapeptide Argireline R marketed by Lipotec, certain carbonyl secondary and tertiary amines, adenosine, and also sapogenins and natural extracts, in particular of Wild Yam, containing them, and also extracts of Boswellia serrata.

Tensioning Agents:

The term “tensioning agent” means a compound capable of exerting tension on the skin, the effect of which is to temporarily fade out irregularities on the skin's surface, such as wrinkles and fine lines.

Among the tensioning agents that may be included in the compositions according to the present invention, especially exemplary are:
(1) synthetic polymers, such as polyurethane latices or acrylic-silicone latices, in particular those described in EP-1-038,519, such as a propylthio(polymethyl acrylate), propylthio(polymethyl methacrylate) and propylthio(polymethacrylic acid) grafted polydimethylsiloxane, or, alternatively, a propylthio(polyisobutyl methacrylate) and propylthio(polymethacrylic acid) grafted polydimethylsiloxane. Such grafted silicone polymers are marketed especially by 3M under the trademarks VS 80, VS 70 or LO21.
(2) polymers of natural origin, especially (a) polyholosides, for example (i) in the form of starch derived especially from rice, corn, potato, cassava, pea, Triticum aestivum wheat, oat, etc. or (ii) in the form of carrageenans, alginates, agars, gellans, cellulose-based polymers and pectins, advantageously as an aqueous dispersion of gel microparticles, and (b) latices consisting of shellac resin, sandarac gum, dammar resins, elemi gums, copal resins and cellulose-based derivatives, and mixtures thereof,
(3) plant proteins and protein hydrolysates, in particular from corn, rye, Triticum aestivum wheat, buckwheat, sesame, spelt, pea, bean, lentil, soybean and lupin,
(3) mixed silicates, especially phyllosilicates and in particular Laponites,
(4) wax microparticles selected, for example, from carnauba wax, candelilla wax and alfalfa wax,
(5) colloidal particles of mineral filler with a number-average diameter of from 0.1 to 100 nm and preferably from 3 to 30 nm, selected, for example, from: silica, silica-alumina composites, cerium oxide, zirconium oxide, alumina, calcium carbonate, barium sulfate, calcium sulfate, zinc oxide and titanium dioxide.

Agents for Stimulating the Synthesis of the Extracellular Matrix:

In particular, the C-glycoside derivatives as described in WO 02/051 828 corresponding to formula (I) below: 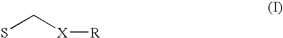
in which,
S represents a monosaccharide or a polysaccharide up to 20 sugar units, in pyranose and/or furanose form and of L and/or D series, the said monosaccharide or polysaccharide containing at least one hydroxyl function 30 that is necessarily free and/or optionally one or more optionally protected amine functions,
the bond S—CH₂X represents a bond of anomeric-C nature,
X represents a group selected from: —CO—, —CH(NR₁R₂)—, —CHR′—, —C(═CHR′)—,
R represents a linear or branched, saturated or unsaturated alkyl, perfluoroalkyl or hydrofluoroalkyl chain, a cycloalkyl, cycloperfluoroalkyl or cyclohydrofluoroalkyl ring containing from 1 to 18 carbon atoms, a phenyl or benzyl radical, the said chain, the said ring or the said radical optionally being interrupted with one or more hetero atoms selected from oxygen, sulfur, nitrogen and silicon, and optionally substituted with at least one radical selected from —OR′₁, —SR″₁, —NR′″₁,R′₂, —COOR″₂, —CONHR′″₂, —CN, halogen, perfluoroalkyl and hydrofluoroalkyl and/or at least one optionally substituted cycloalkyl, aryl or heterocyclic radical,
R′, R₁ and R₂, which may be identical or different, have the same definition as that given for R, and may also represent a hydrogen and a hydroxyl radical,
R′₁, R′₂, R″₁, R″₂, R′″₁ and R′″₂, which may be identical or different, represent a hydrogen atom or a radical selected from a linear or branched, saturated or unsaturated alkyl, hydroxyl, perfluoroalkyl and/or hydrofluoroalkyl radical containing from 1 to 30 carbon atoms.

The photoprotective agents or UV-A and/or UV-B screening agents are in the form of organic or mineral compounds, the latter being optionally coated to make them hydrophobic.

As examples of UV-A-active and/or UV-B-active photoprotective agents, representative are the following, denoted hereinbelow under their INCI name:
para-aminobenzoic acid derivatives, including the following: PABA, Ethyl PABA, Ethyl dihydroxypropyl PAPA, Ethylhexyl dimethyl PABA marketed in particular under the name “Escalol 507” by ISP, Glyceryl PABA, PEG-25 PABA marketed under the name “Uvinul P25” by BASF;
salicylic derivatives, including the following: Homosalate marketed in particular under the name “Neo Heliopan OS” by Haarmann and Reimer, Dipropylene glycol salicylate marketed in particular under the name “Dipsal” by Scher, TEA salicylate marketed in particular under the name “Neo Heliopan TS” by Haarmann and Reimer;
dibenzoylmethane derivatives, including the following: Butyl methoxydibenzoylmethane marketed in particular under the trademark “Parsol 1789” by Hoffmann LaRoche, Isopropyidibenzoylmethane;
cinnamic derivatives, including the following: Ethylhexyl methoxycinnamate marketed in particular under the trademark “Parsol MCX” by Hoffmann LaRoche, Isopropyl methoxycinnamate, Isoamyl methoxycinnamate marketed in particular under the trademark “Neo Heliopan E 1000” by Haarmann and Reimer, Cinoxate, DEA methoxycinnamate, Diisopropyl methylcinnamate, Glyceryl ethylhexanoate dimethoxycinnamate;
β,β′-diphenyl acrylate derivatives, including the following: Octocrylene marketed in particular under the trademark “Uvinul N539” by BASF, Etocrylene marketed in particular under the trademark “Uvinul N35” by BASF;
benzophenone derivatives, including the following: Benzophenone-1 marketed in particular under the trademark “Uvinul 400” by BASF, Benzophenone-2 marketed in particular under the trademark “Uvinul D50” by BASF, Benzophenone-3 or Oxybenzone marketed in particular under the trademark “Uvinul M40” by BASF, Benzophenone-4 marketed in particular under the trademark “Uvinul MS40” by BASF, Benzophenone-5, Benzophenone-6 marketed in particular under the trademark “Helisorb 11” by Norquay, Benzophenone-8 marketed in particular under the trademark “Spectra-Sorb UV-24” by American Cyanamid, Benzophenone-9 marketed in particular under the trademark “Uvinul DS-49” by BASF, Benzophenone-12, and n-hexyl 2-(4-diethylamino-2-hydroxybenzoyl)benzoate;
benzylidenecamphor derivatives, including the following: 3-Benzylidenecamphor, 4-Methylbenzylidenecamphor marketed in particular under the name “Eusolex 6300” by Merck, Benzylidenecamphorsulfonic acid, Camphor benzalkonium methosulfate, Terephthalylidenedicamphorsulfonic acid, Polyacrylamidomethylbenzylidenecamphor;
benzimidazole derivatives, including the following: Phenylbenzimidazolesulfonic acid marketed in particular under the trademark “Eusolex 232” by Merck, Disodium phenyldibenzimidazoletetrasulfonate marketed in particular under the trademark “Neo Heliopan AP” by Haarmann and Reimer;
triazine derivatives, including the following: Anisotriazine marketed in particular under the trademark “Tinosorb S” by Ciba Specialty Chemicals, Ethylhexyltriazone marketed in particular under the trademark “Uvinul T150” by BASF, Diethylhexylbutamidotriazone marketed in particular under the trademark “Uvasorb HEB” by Sigma 3V, and 2,4,6-tris(diisobutyl 4′-aminobenzalmalonate)-s-triazine;
benzotriazole derivatives, including the following: Drometrizole trisiloxane marketed under the name “Silatrizole” by Rhodia Chimie, Methylenebis(benzotriazolyl)tetramethylbutylphenol marketed in particular in solid form under the trademark “MIXXIM BB/100” by Fairmount Chemical, or in micronized form as an aqueous dispersion under the trademark “Tinosorb M” by Ciba Specialty Chemicals;
anthranilic derivatives, including Menthyl anthranilate marketed under the trademark “Neo Heliopan MA” by Haarmann and Reimer;
imidazoline derivatives, including Ethylhexyldimethoxybenzylidenedioxoimidazoline propionate;
benzalmalonate derivatives, including polyorganosiloxane with benzalmalonate functions, marketed under the trademark “Parsol SLX” by Hoffmann LaRoche;
4,4-diarylbutadiene derivatives, including 1,1-dicarboxy(2,2′-dimethylpropyl) 4,4-diphenylbutadiene;
and mixtures thereof.

The organic photoprotective agents that are more particularly preferred are selected from the following compounds: Ethylhexyl salicylate, Ethylhexyl methoxycinnamate, Octocrylene, Phenylbenzimidazolesulfonic acid, Benzophenone-3, Benzophenone4, Benzophenone-5,4-Methylbenzylidenecamphor, Terephthalylidenedicamphorsulfonic acid, Disodium phenyldibenzimidazoletetrasulfonate, 2,4,6-Tris(diisobutyl 4′-aminobenzalmalonate)-s-triazine, Anisotriazine, Ethylhexyltriazone, Diethylhexylbutamidotriazone, Methylenebis(benzotriazolyl)tetramethylbutylphenol, Drometrizole trisiloxane, 1,1-dicarboxy(2,2′-dimethylpropyl)-4,4-diphenylbutadiene and mixtures thereof.

The mineral photoprotective agents are selected from pigments or even nanopigments (mean size of the primary particles: generally from 5 nm to 100 nm and preferably from 10 nm to 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are described in particular in EP-518,772 and EP-518,773.

The photoprotective agents are generally present in the compositions according to the invention in proportions ranging from 0.1% to 20% by weight relative to the total weight of the composition and preferably ranging from 0.2% to 15% by weight relative to the total weight of the composition.

It is also advantageously possible to include in the compositions for caring for and/or making up the skin or the lips according to the invention, makeup agents with an optical effect that enhances the appearance of the skin or the lips, and/or the skin complexion.

The amount of 8-hexadecene-1,16-dicarboxylic acid may range from 0.00001% to 5% or from 0.00001% to 8% by weight relative to the total weight of the composition, and amounts suited to the use of the invention will especially be from 0.0001% to 0.005% or from 0.0001% to 0.0005% by weight relative to the total weight of the composition.

In particular, this makeup agent with an optical effect that enhances the appearance and/or complexion of the skin is selected from soft-focus fillers, fluorescent agents and optical brighteners, and mixtures thereof.

The term “soft-focus” filler means a filler which in addition gives the complexion transparency and a hazy effect. Preferably, the soft-focus fillers have a mean particle size of less than or equal to 15 microns. These particles may be in any form and in particular may be spherical or non-spherical. These fillers are more preferably non-spherical.

The soft-focus fillers may be selected from silica and silicate powders, especially alumina powder, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Mention may be made in particular of talc with a number-average size of less than or equal to 3 microns, for example talc with a number-average size of 1.8 microns and especially the product marketed under the trademark Talc P3® by Nippon Talc, Nylon® 12 powder, especially the product marketed under the name Orgasol 2002 Extra D Nat Cos® by Atochem, silica particles 1% to 2% surface-treated with a mineral wax (INCI name: hydrated silica (and) paraffin) such as the products marketed by Degussa, amorphous silica microspheres, such as the products marketed under the name Sunsphere, for example of reference H-53 by Asahi Glass, and silica microbeads such as those marketed under the name SB-700® or SB-150® by Miyoshi, this list not being limiting.

The soft-focus filler may be present in the cosmetic compositions with a soft-focus effect in a content ranging from 0.1% to 20% by weight, especially ranging from 1% to 12% by weight and especially from 5% to 10%, for example about 8%, by weight relative to the total weight of the composition.

The term “fluorescent agent” means a substance which, under the effect of ultraviolet rays and/or visible light, re-emits in the visible region the portion of light that it has absorbed under the same color as that which it naturally reflects. The naturally reflected color is thus reinforced by the re-emitted color and appears extremely bright.

Examples that are representative include colored polyamide and/or formaldehyde/benzoguanamine and/or melamine/formaldehyde/sulfonamide resins, from colored aminotriazine/formaldehyde/sulfonamide co-condensates and/or from metallized polyester flakes and/or mixtures thereof. These fluorescent pigments may also be present in the form of aqueous dispersions of fluorescent pigments.

Mention may also be made of the pink-colored fluorescent aminotriazine/formaldehyde/sulfonamide co-condensate with a mean particle size of 3-4 microns marketed under the trademark “Fiesta Astral Pink FEX-1” and the blue-colored fluorescent aminotriazine/formaldehyde/sulfonamide co-condensate with a mean particle size of 3-4.5 microns marketed under the trademark “Fiesta Comet Blue FTX-60” by Swada, or, alternatively, the yellow-colored benzoguanamine/ formaldehyde resin covered with formaldehyde/urea resin marketed under the trademark “FB-205 Yellow” and the red-colored benzoguanamine/formaldehyde resin covered with formaldehyde/urea resin marketed under the trademark “FB-400 Orange Red” by UK Seung Chemical, and the orange-colored polyamide resin marketed under the trademark “Flare 911 Orange 4” by Sterling Industrial Colors.

The fluorescent substances are preferably present in the compositions in a content ranging from 0.1% to 20%, preferably from 0.1% to 15% to more preferably from 0.5% to 3% by weight relative to the total weight of the composition.

When the organic fluorescent substances are white, they are also known as optical brighteners.

The optical brightener has the effect of intensifying the radiance and reviving the shades of cosmetic compositions comprising them on application to the skin.

Among the optical brighteners that are more particularly representative are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trademarks Tinopal SOP® and Uvitex OB® from the company Ciba Geigy.

The optical brighteners preferentially employed are sodium 4,4′-bis[(4,6-dianilino-1,3,5-triazin-2-yl)amino]stilbene-2,2′-disulfonate, 2,5-thiophenediylbis(5-tert-butyl-1,3-benzoxazole) and disodium 4,4′-distyrylbiphenylsulfonate, and/or mixtures thereof.

This invention also features the use of 8-hexadecene-1,16-dicarboxylic acid in a process for preparing reconstructed epidermis and/or reconstructed skin, or a model of hair follicle in survival or of hornified epithelium, as an agent for promoting the cohesion, correct formation and strength of the horny layer of the said reconstructed epidermis and/or reconstructed skin. The step of forming the horny layer is an intricate step in a process of epidermal reconstruction. It is thus advantageous to use a compound capable of promoting good formation and cohesion of the horny layer in such a process.

These reconstructed epidermis and/or reconstructed skin may be used as models for screening and/or evaluating cosmetic or dermatological active agents, or may be intended for treating individuals with damaged skin (major burns; skin excisions; patients suffering from a genetic disease affecting the skin, such as bulbous epidermolysis, Xeroderma pigmentosum, lamellar ichtyosis and ichtyosis associated with the X chromosome).

The process for preparing reconstructed epidermis and/or reconstructed skin conventionally comprises:
a) a step of preparing a support or a dermal equivalent; and
b) a step of inoculating the said support with a population of human keratinocytes.

8-Hexadecene-1,16-dicarboxylic acid may be added to the culture medium of the said cells, in one and/or the other of the above-described steps. The amount of 8-hexadecene-1,16-dicarboxylic acid present in the culture medium may range from 0.001 μg/ml to 20 μg/ml, preferably from 0.01 μg/ml to 5 μg/ml and even more preferentially from 0.1 μg/ml to 1 μg/ml.

In particular, the said support or dermal equivalent will be selected from collagen/fibroblast latices, a dermis that has been de-epidermidized beforehand and artificial membranes. The protocols described in (EP-A-285,471, EP-A-285,474, EP-A-789,074, EP-A-502,172, EP-A-418,035, WO-A-91/16010, EP-A-197 090, EP-A-20753, FR-A-2 665 175, FR-A-2 689 904) or, preferably, the protocol described by Asselineau et al.,1987 (Models in Dermato., vol. III, Ed. Lowe & Maibach, 1-7) may be mentioned as examples of preparation of a dermal equivalent.

Examples of protocols for preparing epidermal and/or skin equivalents that may be mentioned are those described in EP-285,471, EP-285,474, EP-418,035, WO-A-90/02796, WO-A-91/16010, EP-197,090, EP-20753, FR-2-665,175 and FR-2-689,904.

In very general terms, the models of reconstructed skin consist of human keratinocytes deposited on a support, usually a dermal equivalent, and cultured under conditions such that they begin a programme of differentiation resulting in the formation of an epidermal equivalent.

Other cell types such as Langerhans cells (EP 0 789 074) or melanocytes may also be incorporated, to reconstitute an epidermis and/or a skin similar to native tissues.

The present invention also features a skin or lip makeup composition comprising, in a physiologically acceptable medium, 8-hexadecene-1,16-dicarboxylic acid in an amount ranging from 0.00001% to 3% by weight relative to the total weight of the composition and characterized in that it is a tinted cream, a foundation, a lipstick, a lip gloss, a lip contour pencil or a mascara.

The amount of 8-hexadecene-1,16-dicarboxylic acid will preferably be greater than or equal to 0.0001%; amounts suited to the use of the invention will especially be from 0.0001% to 0.005% or from 0.0001% to 0.0005% by weight relative to the total weight of the composition.

This invention also features cosmetic compositions comprising, in a physiologically acceptable medium, (a) at least 8-hexadecene-1,16-dicarboxylic acid and (b) at least one skin-coloring agent.

More particularly, the skin-coloring agent is selected from DHA and polyphenols.

The present invention also features compositions comprising, in a physiologically acceptable medium, (a) at least 8-hexadecene-1,16-dicarboxylic acid and (b) one other agent for promoting the homogeneity of the horny layer, such as urea.

The composition may be in the form of an optionally gelled aqueous, aqueous-alcoholic or oily solution, an emulsion of liquid or semi-liquid consistency of the milk type, obtained by dispersing a fatty phase in an aqueous phase (O/W) or conversely (W/O), a triple emulsion (W/O/W or O/W/O), or a suspension or emulsion of soft, semi-solid or solid consistency of cream or gel type, a liquid, pasty or solid anhydrous product, or, alternatively, microemulsions, microcapsules, microparticles or a vesicular dispersion of ionic type (liposomes or oleosomes) and/or nonionic type (niosomes) and/or a dispersion of nanospheres.

A composition in mousse form or in spray or aerosol form then comprising a pressurized propellant is also intended.

The composition may thus be in the form of a lotion, serum, milk, O/W or W/O cream, gel, ointment, pomade, powder, balm, patch, impregnated pad, soap, bar or mousse. It may also be in the form of a lipstick, a lip paste or a lip gloss, a powder, or a solid or semi-solid foundation.

Advantageously, the skin or lip makeup composition may also comprise at least one cosmetic adjuvant selected from fillers, dyestuffs, hydrophilic or lipophilic cosmetic active agents, thickeners, emulsifiers, hydrophilic or lipophilic gelling agents, surfactants, moisturizers, softeners, sequestrants, fragrances, neutralizers, preservatives, antioxidants, UV-screening agents, bactericides, trace elements, odor absorbers and pH regulators, and mixtures thereof.

In particular, the cosmetic active agent may especially be selected from an agent for stimulating the synthesis of dermal or epidermal macromolecules and/or for preventing their degradation, an agent for stimulating fibroblast or keratinocyte proliferation and/or keratinocyte differentiation, a moisturizer, a depigmenting agent, a skin-coloring agent, an anti-pollution agent or free-radical scavenger, a dermo-relaxing agent and a tensioning agent, and mixtures thereof.

Examples of such compounds are described previously.

It may also comprise at least one makeup agent with an optical effect that enhances the skin and/or the lips, in particular a complexion-enhancing optical effect.

In particular, this makeup agent with an optical effect that enhances the appearance and/or complexion of the skin is selected from soft-focus fillers, fluorescent agents and optical brighteners, and mixtures thereof.

Examples of such compounds are described previously.

This invention also features cosmetic or dermatological compositions comprising, in a physiologically acceptable medium, 8-hexadecene-1,16-dicarboxylic acid and at least one acid selected from lactic acid, glycolic acid and ascorbic acid in a quantitative ratio ranging from 0.005:1 to 0.05:1 and preferably a ratio of 0.01:1.

In particular, the composition contains glycolic acid, 8-hexadecene-1,16-dicarboxylic acid and ascorbic acid in a 1:0.01:1 ratio.

Preferably, the 8-hexadecene-1,16-dicarboxylic acid is present in the composition in an amount ranging from 0.0001% to 0.1% by weight relative to the total weight of the composition.

This composition may especially be used in a scrubbing process with a preventive effect on impairment of the formation of the horny layer or a repairing effect (sequential or simultaneous scrubbing and repairing twofold effect).

In one particular embodiment, the invention features kits comprising a first composition comprising at least one scrubbing agent and a second composition comprising at least 8-hexadecene-1,16-dicarboxylic acid in an amount ranging from 0.00001% to 0.005% by weight relative to the total weight of the second composition.

In particular, the scrubbing agent is selected from keratolytic agents or desquamating agents, and the keratolytic agents are selected from α-hydroxy acids; β-hydroxy acids; α-keto acids; β-keto acids; the retinoids and desquamating agents are selected from β-hydroxy acids; α-hydroxy acids; urea; gentisic acid; oligofucoses; cinnamic acid; extract of Saphora japonica; resveratrol and certain jasmonic acid derivatives; mineral-salt chelating agents: EDTA; N-acyl-N,N′,N′-ethylenediaminetriacetic acid; aminosulfonic compounds and in particular (N-2-hydroxyethylpiperazine-N-2-ethane)sulfonic acid (HEPES); 2-oxothiazolidine-4-carboxylic acid (procysteine) derivatives; derivatives of α-amino acids of glycine type (as described in EP-0 852 949, and also sodium methyl glycine diacetate marketed by BASF under the trademark Trilon M); honey; sugar derivatives such as O-octanoyl-6-D-maltose and N-acetylglucosamine.

The second composition may also comprise a compound selected from glycolic acid, lactic acid and ascorbic acid, more specifically such that the 8-hexadecene-1,16-dicarboxylic acid and the other acid selected from lactic acid, glycolic acid and ascorbic acid, and mixtures thereof, are present in the composition in a quantitative ratio ranging from 0.005:1 to 0.05:1 and preferably in a ratio of 0.01:1.

Finally, this second composition may also comprise urea, the concentration of which may be from 0.1% to 10% by weight.

In the process according to the invention, it is possible to perform the steps comprising:
(i) applying at least one desquamating agent at a concentration and for a time that induces scrubbing and/or desquamating activity,
(ii) applying at least 8-hexadecene-1,16-dicarboxylic acid or a composition containing it at least a concentration that is effective for improving the cohesion and/or formation of the horny layer as defined previously.

Steps (i) and (ii) may be simultaneous or staggered; in this second case, they may be inverted and/or repeated over time.

The desquamating agent of step (i) is as defined previously. It may be selected especially from the group comprising salicylic acid and derivatives thereof such as 5-n-octanoylsalicylic acid described in WO 04/073 605, sulfonic acids, calcium-chelating agents, α-hydroxy acids such as glycolic acid, citric acid, lactic acid, tartaric acid, malic acid or mandelic acid; ascorbic acid and derivatives thereof such as ascorbyl glucoside and magnesium ascorbyl phosphate; nicotinic acid and nicotinamide; urea; and (N-2-hydroxyethylpiperazine-N-2-ethane)sulfonic acid (HEPES), β-hydroxy acids such as salicylic acid and derivatives thereof, retinoids such as retinol and esters thereof, retinal, retinoic acid and derivatives thereof, those described in FR-2-570,377, EP-199,636, EP-325,540 and EP402,072, extracts of chestnut or of Barbary fig, in particular marketed by Silab; reducing compounds such as cysteine or cysteine precursors.

The present invention also features a cosmetic regime or regimen for improving the surface appearance and/or the comfort of the skin, the scalp or the lips, comprising the topical application to the skin, the scalp or the lips of a care or makeup composition as defined above.

In particular, the cosmetic regime or regimen according to the invention is suited for improving the homogeneity of the complexion and/or for promoting the staying power of a composition on the skin or the lips.

The compositions according to the invention may be applied daily to the lips or to the whole face to obtain a uniform complexion.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Effect on the Formation of the Horny Layer

Choice of Concentrations:

Each test product is applied for 24 hours at different concentrations to normal human epidermal keratinocytes (NHEK) K015 in culture, and the non-irritant and non-cytotoxic maximum dose for the said cells is evaluated. This evaluation is made by visual analysis of the cell carpets and reduction of MTT [3-(4,5-dimethylthiazol-2-yl)-2,5-diphenyltetrazolium bromide], which is representative of the cell viability (Mosman T, Immunol. Methods, 1983, 65: 55-63).

The non-cytotoxic effective doses are thus obtained.

These active agents were then tested at the non-irritant and non-cytotoxic doses thus defined for their capacity to promote correct formation of the horny layer.

Analysis of the Differential expression by Minichips:

In order to study the activity of a product on the formation of the horny layer, the effect of the product on the expression of genes known to be modulated in keratinocytes during their transformation into corneocytes is analyzed.

The analysis is performed using dedicated minichips, consisting of cDNA membrane arrays known to those skilled in the art for their relationship with re-epithelialization and/or cohesion of the horny layer, especially such as the LEP proteins (late epidermal proteins), corneodesmosines and epidermal growth factor HBEGF (human heparin-binding EGF-like growth factor).

The cell model used comprises normal human epidermal keratinocytes (NHEK). The keratinocytes are cultured in SFM medium with 0.25 ng/ml EGF and 25 μg/ml of pituitary extract at 37° C. and 5% CO₂. The keratinocytes are inoculated in 25 cm² dishes and precultured in whole SFM medium, and then placed in EGF-free and pituitary extract-free SFM medium.

The test products are applied at the non-irritant and non-cytotoxic concentrations defined in a).

The controls correspond to the same conditions, in the absence of product.

The cells are then rinsed with PBS and then lysed and the messenger RNA is extracted and purified using the kit Chemagic mRNA Direct kit marketed by Chemagen.

The RNA of each culture is then reverse-transcripted using a pool of primers corresponding to the cDNAs immobilized on the membranes (probes) and a ³³P-labelled deoxynucleotide triphosphate.

Multiple cDNA “target” labeled sequences were thus prepared for each culture. These targets were then hybridized, under optimal conditions, with the excess “probe” cDNAs bound to the membranes. After washing, the relative amount of labeled target was revealed by autoradiography and by direct counting on a Phosphorimager. The membrane analysis was performed using the ImageQuant TL software (Image Analysis, Amersham Biosciences) and the data recorded were then processed on Excel. The differential expression of the various genes following the treatment is thus measured by comparison with the corresponding control.

The results presented in the table below are expressed by a multiplicative factor demonstrating the stimulatory activity of the test products on the expression of genes known to be involved in the formation of the horny layer.

| | Markers for the formation | |
|---|---|---|
| | of the horny layer (LEP) | Re- |
| Test active agents | and for intercorneocytic | epithelialization |
| (concentration) | cohesion (Cdsn) | markers |
| Lactic acid | LEP16 (×1.6) | — |
| (100 μg/ml) | | |
| Glycolic acid | Cdsn (×1.6) | — |
| (100 μg/ml) | LEP16 (×2.6) | |
| Dioic acid* | Cdsn (×2.4) | HBEGF (×1.6) |
| (1 μg/ml) | LEP16 (×3.0) | |
| Ascorbic acid | Cdsn (×1.7) | — |
| (100 μg/ml) | | |

*= 8-hexadecene-1,16-dicarboxylic acid

The results show that 8-hexadecene-1,16-dicarboxylic acid, at low concentration (1 μg/ml), is capable of stimulating the expression of the genes for LEP16, corneodesmosine and HBEGF.

These effects are also obtained for lactic acid, glycolic acid and ascorbic acid at concentrations of 100 μg/ml.

These transcriptomic results were confirmed by quantitative RT-PCR.

Quantitative RT-PCR:

The pairs of primers (markers) used in this study allow the amplification of the following specific fragments:
LEP16 under the No. GenBank NM_{—}032563;
HBEGF (human heparin-binding EGF-like growth factor) under the No. GenBank M60278;
CDSN (corneodesmosin precursor) under the No. GenBank L20815;
actin (human beta-actin) under the No. GenBank X00351, used as control.

The total RNA remaining from the cDNA-array study described in b) is used. The potentially contaminant DNA traces are removed by DNAse treatment and inactivation of DNAse. A reverse transcription reaction is then performed, followed by quantification, by fluorescence, of the cDNA synthesized.

The PCR (polymerase chain reaction) reactions were performed by quantitative PCR with the “Light Cycler” system (Roche Molecular Systems Inc.) and according to the procedures recommended by the manufacturer. A first series of Q-PCR is performed on the β-actin marker (control) to check the homogeneity of the preparations to be compared. Next, Q-PCRs are performed in triplicate using pairs of primers specific for the β-actin sequences, and for the markers to be studied. The fluorescence analysis in the amplified DNA is measured continuously during the PCR cycles. The mean value of the relative expression (RE) is expressed in Arbitrary Units (AU) calculated from the values of two independent PCR cycles according to the following formula: (1/2^{number of cycies})×106.

The results for the differential expression of 3 markers LEP16, HBEGF and Cdsn are compared with that for β-actin.

The increase in the relative expression of the LEP16, HBEGF and Cdsn markers visualized according to the cDNA-array method described in b) is confirmed by RT-PCR for the test products.

These results as a whole show that 8-hexadecene-1,16-dicarboxylic acid at low concentration is capable of promoting and/or restoring the formation and cohesion of the horny layer.

It is advantageous to use it in compositions for caring for and/or making up the skin or the lips, in hair-shaping compositions, and also for the preparation of dermatological compositions for treating skin pathologies associated with abnormal formation of the horny layer.

Moreover, it will be more advantageous to combine it with at least one acid selected from lactic acid, glycolic acid and ascorbic acid to optimize and/or complement the desired effect (maturation of the horny layer or re-epithelialization).

### EXAMPLE 2

### Formulations

Regenerating Care Cream:

| | |
|---|---|
| 8-hexadecene-1,16-dicarboxylic acid | 0.001% |
| glycolic acid | 0.01% |
| ascorbic acid | 0.1% |
| methylparaben | 0.1% |
| propylparaben | 0.1% |
| lanolin | 5% |
| liquid petroleum jelly | 4% |
| sesame seed oil | 4% |
| cetyl alcohol | 5% |
| glyceryl monostearate | 2% |
| triethanolamine | 1% |
| propylene glycol | 5% |
| carbomer 940 | 0.1% |
| water | qs 100% |

Foundation:

| | |
|---|---|
| 8-hexadecene-1,16-dicarboxylic acid | 0.001% |
| microcrystalline wax | 3% |
| isostearyl neopentanoate | 15% |
| isononyl isononanoate | 10% |
| carnauba wax | 2% |
| polydimethylsiloxane (viscosity 10 cSt) | 25% |
| zinc oxide | 2% |
| titanium oxide | 12.5% |
| yellow iron oxide | 3.5% |
| black iron oxide | 0.5% |
| preservative | 0.2% |
| kaolin | 3% |
| silica microspheres | 8.11% |
| sericite (BC281 from Whittaker) | 10% |
| nanotitanium | 2% |

Such a composition is in the form of a cast anhydrous compact.

Erythemal Care Cream:

| | |
|---|---|
| 8-hexadecene-1,16-dicarboxylic acid | 0.004% |
| glyceryl stearate | 2.00% |
| polysorbate 60 | 1.00% |
| stearic acid | 1.40% |
| glycyrrhetinic acid | 2.00% |
| triethanolamine | 0.70% |
| carbomer | 0.40% |
| extract of Aloe vera | 2.00% |
| sunflower oil | 10.00% |
| antioxidant | 0.05% |
| fragrance | 0.50% |
| preservative | 0.30% |
| water | qs 100% |

Composition with a Twofold Effect: Scrubbing and Post-Scrubbing Repair:

| | |
|---|---|
| 8-hexadecene-1,16-dicarboxylic acid | 0.01% |
| glycolic acid | 1% |
| mandelic acid | 1% |
| water | 20% |
| polyethylene glycol | qs 100% |

Lipstick:

| | |
|---|---|
| 8-hexadecene-1,16-dicarboxylic acid | 0.003% |
| polyethylene wax (MW 500) | 12.00% |
| liquid lanolin | 15.00% |
| phenyl trimethicone (DC556 from Dow Corning) | 63.34% |
| pigments | 8.66% |

Hair Lotion for Improving the Sheen and hold of the Hair and also the Appearance of the Scalp at the Hair Root Junction:

| | |
|---|---|
| 8-hexadecene-1,16-dicarboxylic acid | 0.1% |
| 2,4-DPO | 1.5% |
| valine | 0.01% |
| arginine | 0.01% |
| ethanol | 40% |
| propylene glycol | 5% |
| water | qs 100 |

Hair Lotion for Promoting Hair Growth and also the Appearance of the Scalp at the Hair Root Junction:

| | |
|---|---|
| 8-hexadecene-1,16-dicarboxylic acid | 0.01% |
| tripeptide KPV | 0.005% |
| arginine | 0.01% |
| taurine | 0.01% |
| ethanol | 40% |
| propylene glycol | 6% |
| water | qs 100 |

Lotion for Improving the Appearance of the Nail Contour:

| | |
|---|---|
| 8-hexadecene-1,16-dicarboxylic acid | 0.5% |
| arginine | 0.01% |
| ceramide-R | 0.001% |
| cysteine B6 | 0.0001% |
| taurine | 0.001% |
| propylene glycol | 10% |
| ethanol | 20% |
| water | qs 100 |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for promoting cohesion and/or organization of the epidermal horny layer, comprising administering to an individual in need of such treatment, for such period of time as required to elicit the desired effect, a cosmetic/dermatological composition which comprises up to 8% by weight of 8-hexadecene-1,16-dicarboxylic acid, formulated into a physiologically acceptable medium therefor.

2. The regime or regimen for promoting cohesion and/or organization of the epidermal horny layer as defined by claim 1, said cosmetic/dermatological composition comprising from 0.00001% to less than 0.005% by weight of said 8-hexadecene-1,16-dicarboxylic acid.

3. The regime or regimen for promoting cohesion and/or organization of the epidermal horny layer as defined by claim 1, including promoting the homogeneity and/or clarity of the complexion.

4. The regime or regimen for promoting cohesion and/or organization of the epidermal horny layer as defined by claim 1, including promoting the homogeneity and/or staying power of a makeup composition applied to the skin or the lips.

5. The regime or regimen for promoting cohesion and/or organization of the epidermal horny layer as defined by claim 1, including preventing impairment of said horny layer induced by a cosmetic or dermatological active agent.

6. The regime or regimen for promoting cohesion and/or organization of the epidermal horny layer as defined by claim 1, including promoting re-epithelialization and/or regeneration of the skin or the lips and of the contour of the hair root or of the nails.

7. The regime or regimen for promoting cohesion and/or organization of the epidermal horny layer as defined by claim 1, including improving the comfort of the skin, the scalp, the nails or the lips, impaired by at least one condition selected from among cold, UV radiation and mechanical rubbing.

8. A regime or regimen for unifying the color of a suntan, comprising administering to an individual in need of such treatment, for such period of time as required to elicit the desired effect, a cosmetic/dermatological composition which comprises up to 8% by weight of 8-hexadecene-1,16-dicarboxylic acid, formulated into a physiologically acceptable medium therefor.

9. A cosmetic/dermatological composition useful for preventing impairment of cohesion of the epidermal horny layer and/or for promoting restoration thereof, comprising a horny layer cohesion-impairing amount of at least one cosmetic/dermatological active agent and an amount of 8-hexadecene-1,16-dicarboxylic acid effective to counter the adverse effects of said at least one active agent, formulated into a physiologically acceptable medium therefor.

10. The cosmetic/dermatological composition as defined by claim 9, said at least one such cosmetic/dermatological active agent being selected from the group consisting of keratolytic or desquamating agents, anti-seborrhoeic and/or anti-acne agents and comedolytic agents, and mixtures thereof.

11. A regime or regimen for treating a skin disorder, condition or affliction associated with abnormal maturation of the epidermal horny layer, comprising administering to an individual in need of such treatment, for such period of time as required to elicit the desired effect, a cosmetic/dermatological composition which comprises a thus effective amount of 8-hexadecene-1,16-dicarboxylic acid, formulated into a physiologically acceptable medium therefor.

12. The regime or regimen as defined by claim 11, said skin disorder, condition or affliction associated with abnormal maturation of the epidermal horny layer being selected from the group consisting of hyperkeratosis, parakeratosis, leukokeratosis and cutaneous trophic disorders.

13. The regime or regimen as defined by any of claims 8, 11 or 12, said cosmetic/dermatological composition comprising from 0.00001% to less than 0.005% by weight of said 8-hexadecene-1,16-dicarboxylic acid.

14. The cosmetic/dermatological composition as defined by claims 9 or 10, comprising from 0.00001% to less than 0.005% by weight of said 8-hexadecene-1,16-dicarboxylic acid.

15. A cosmetic/dermatological composition useful for promoting cohesion and/or organization of the epidermal horny layer, comprising thus effective amounts of 8-hexadecene-1,16-dicarboxylic acid in combination with at least one other acid selected from the group consisting of lactic acid, glycolic acid, ascorbic acid, and mixtures thereof, formulated into a physiologically acceptable medium therefor.

16. The cosmetic/dermatological composition as defined by claim 15, said 8-hexadecene-1,16-dicarboxylic acid and said at least one other acid being present therein in a quantitative ratio ranging from 0.005:1 to 0.05:1.

17. The cosmetic/dermatological composition as defined by claim 16, said quantitative ratio being about 0.01:1.

18. A regime or regimen for promoting cohesion and/or organization of the epidermal horny layer, comprising administering to an individual in need of such treatment, for such period of time as required to elicit the desired effect, a cosmetic/dermatological composition which comprises thus effective amounts of 8-hexadecene-1,16-dicarboxylic acid in combination with at least one other acid selected from the group consisting of lactic acid, glycolic acid, ascorbic acid, and mixtures thereof, formulated into a physiologically acceptable medium therefor.

19. The cosmetic/dermatological composition as defined by claim 15, further comprising urea.

20. The regime or regimen as defined by claim 18, said cosmetic/dermatological composition further comprising urea.

21. A kit comprising a first composition comprising at least one scrubbing agent and a second composition comprising at least 8-hexadecene-1,16-dicarboxylic acid in an amount ranging from 0.00001% to 0.005% by weight relative to the total weight of the second composition.

22. The kit as defined by claim 21, said at least one scrubbing agent being selected from the group consisting of keratolytic agents and desquamating agents.

23. The kit as defined by claim 22, comprising at least one keratolytic agent selected from the group consisting of α-hydroxy acids; β-hydroxy acids; α-keto acids; β-keto acids; the retinoids and/or at least one desquamating agent selected from the group consisting of β-hydroxy acids; α-hydroxy acids; urea; gentisic acid; oligofucoses; cinnamic acid; extract of Saphora japonica; resveratrol and jasmonic acid derivatives; mineral-salt chelating agents: EDTA; N-acyl-N,N′,N′-ethylenediaminetriacetic acid; aminosulfonic compounds; (N-2-hydroxyethylpiperazine-N-2-ethane)sulfonic acid (HEPES); 2-oxothiazolidine-4-carboxylic acid (procysteine) derivatives; derivatives of α-amino acids of glycine type; sodium methyl glycine diacetate; honey; sugar derivatives; O-octanoyl-6-D-maltose; N-acetylglucosamine; and mixtures thereof.

24. The kit as defined by claim 21, said second composition further comprising glycolic acid, lactic acid and/or ascorbic acid.

25. The kit as defined by claim 24, said 8-hexadecene-1,16-dicarboxylic acid and the other acid selected from among lactic acid, glycolic acid and ascorbic acid, and mixtures thereof, being present in the composition in a quantitative ratio ranging from 0.005:1 to 0.05:1.

26. The kit as defined by claim 21 or 24, said second composition further comprising urea.

27. A regime or regimen for regenerating and promoting cohesion of the epidermal horny layer, comprising topically applying onto the skin of an individual in need of such treatment, after a scrubbing operation thereon, a cosmetic/dermatological composition which comprises a thus effective amount of 8-hexadecene-1,16-dicarboxylic acid, formulated into a physiologically acceptable medium therefor.

28. A skin or lip makeup composition formulated as a tinted cream, a foundation, a lipstick, a lip gloss, or a lip contour pencil and comprising from 0.00001% to less than 3% by weight of 8-hexadecene-1,16-dicarboxylic acid.

29. The skin or lip makeup composition as defined by claim 28, further comprising at least one soft-focus filler, fluorescent agent, optical brightener, and mixture thereof.

30. A cosmetic/dermatological composition useful for promoting cohesion and/or organization of the epidermal horny layer, comprising a thus effective amount of 8-hexadecene-1,16-dicarboxylic acid and at least one skin-coloring agent, formulated into a physiologically acceptable medium therefor.

31. The cosmetic/dermatological composition as defined by claim 30, said at least one skin-coloring agent comprising DHA and/or a polyphenol.

32. A cosmetic/dermatological composition useful for promoting cohesion and/or organization of the epidermal horny layer, comprising a thus effective amount of 8-hexadecene-1,16-dicarboxylic acid and at least one other agent for promoting cohesion of the horny layer, formulated into a physiologically acceptable medium therefor.

33. The regime or regimen as defined by claim 1, including improving the surface appearance and/or the comfort of the skin, the scalp or the lips, comprising topically applying thereon said cosmetic/dermatological composition.

34. In a process for preparing reconstructed epidermis and/or reconstructed skin and/or hair follicles in survival, the improvement which comprises utilizing as an agent for promoting cohesion and maturation of the horny layer of the said reconstructed epidermis and/or reconstructed skin and/or of the said hair follicles in survival, a thus effective amount of 8-hexadecene-1,16-dicarboxylic acid.

35. A reconstructed epidermis and/or reconstructed skin prepared by the process as defined by claim 34.

36. A regime or regimen for inducing the expression of proteins involved in the cohesion of the epidermal horny layer or intercorneocytic cohesion, comprising administering to an individual in need of such treatment, a thus effective amount of 8-hexadecene-1,16-dicarboxylic acid.

37. A cosmetic/dermatological composition useful for promoting cohesion and/or organization of the epidermal horny layer, comprising a thus effective, non-skin irritating amount of 8-hexadecene-1,16-dicarboxylic acid and at least one other agent selected from the group consisting of agents for stimulating the synthesis of dermal or epidermal macromolecules and/or for preventing their degradation, agents for stimulating fibroblast or keratinocyte proliferation and/or keratinocyte differentiation, moisturizers, depigmenting agents, anti-pollution agents, free radical scavengers, dermo-relaxing agents, tensioning agents, agents for stimulating the synthesis of the extracellular matrix, and mixtures thereof.
